(19) United States
(12) Patent Application Publication
(10) Pub. No.: US 2025/0295826 A1
(43) Pub. Date: Sep. 25, 2025

(54) FRAGRANCES AS UREASE INHIBITORS

(71) Applicant: Symrise AG, Holzminden (DE)

(72) Inventors: Marco SINGER, Colombes (FR); Laura MEUNIER, Holzminden (DE); Katharine PRIGGE, West Caldwell, NJ (US); Keith MC DERMOTT, Bound Brook, NJ (US); Edison DIAZ, Goslar (DE)

(21) Appl. No.: 18/849,022

(22) PCT Filed: Apr. 20, 2022
(86) PCT No.: PCT/EP2022/060394

§ 371 Date: Sep. 20, 2024

Related U.S. Application Data
(60) Provisional application No. 63/322,417, filed on Mar. 22, 2022.

(51) Int. Cl. A61L 9/01; A01K 1/015; A61L 101/32

(52) U.S. Cl. CPC A61L 9/01; A01K 1/0152; A61L 2101/32

(57) ABSTRACT

Suggested is the use of at least one fragrance having a molecular weight between 95 to 225 g/mol as a urease inhibitor.

### AREA OF INVENTION

The present invention relates to the field of functional perfumery, in particular to the prevention of ammonia malodor formation, more particular to fragrances which have the ability to inhibit the enzyme urease.

### BACKGROUND OF THE INVENTION

Fragranced products such as perfumes, deodorants or other fragrance-containing personal care, home care, pet care or cosmetic products have widely been used to mask unpleasant odors. Overcoming ammonia malodors however is a major challenge due to its ability to not only activate the olfactory system but also trigger trigeminal chemo sensation. The latter is known to be a warning system for exposure to chemical hazards (in this case due to changes of pH) and therefore the perception is very unpleasant and hard to mask by conventional fragrances. As a result, the best approach to counteract ammonia odors is to prevent their formation.

The most prevalent form of ammonia formation in living systems and environments is through enzymatic degradation of urea by bacterial urease. As a result, one can either use antibacterial agents to reduce the amount of urease-positive bacteria or chemically inhibit the enzyme urease to prevent the formation of ammonia.

Inhibition of urease has been thoroughly studied from a medicinal point of view but also for agricultural products in particular urea containing fertilizers. In both areas, very powerful small molecule inhibitors were developed that are highly specific and effective against urease. Unfortunately, these compounds are not suitable for use in consumer products for several reasons, e.g. safety and cost aspects.

As a result, it was the object of the invention to provide cost effective, safe and fragrance compatible urease inhibitors that can be readily used in consumer products to tackle the challenge of ammonia malodor formation.

### DESCRIPTION OF THE INVENTION

According to the invention, there is thus provided at least one fragrance having a molecular weight between 95 to 225 g/mol, which is used as a urease inhibitor, particular as a urease inhibitor.

The inventors have surprisingly found that fragrances which have a molecular weight between 95 to 225 g/mol inhibit the activity of urease, in particular bacterial urease, thereby reducing the amount of ammonia generated from ammonia precursors, as for example, urea.

Ureases functionally belong to the superfamily of amidohydrolases and phosphotriesterases. Ureases are found in numerous bacteria, fungi, algae, plants, and some invertebrates, as well as in soils, as a soil enzyme. They are nickel-containing metalloenzymes of high molecular weight. These enzymes catalyze the hydrolysis of urea into carbon dioxide and ammonia:

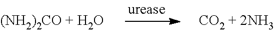

The hydrolysis of urea occurs in two stages. In the first stage, ammonia and carbamate are produced. The carbamate spontaneously and rapidly hydrolyzes to ammonia and carbonic acid. Urease activity increases the pH of its environment as ammonia is produced, which is basic.

The fragrances to be used according to the invention are furthermore advantageously easily accessible or are readily available or can be easy prepared, are highly effective even at low concentrations, in particular at concentrations at which the fragrances have no odor or at least only a barely perceptible odor, are largely or completely colorless, have a high stability in various mixtures or preparations and have no toxic and/or allergenic effect on humans.

The fragrances of the present invention also have the advantage that they can be combined with different fragrances, other than the fragrances according to the invention, and perfume oils or usual components of a perfume oil in order to perfume products with any desired fragrance. Consequently, by the present invention, a wide range of fragrance types can be offered to consumers.

A “urease inhibitor” or “urease inhibitors” according to the present invention are chemical compounds, in particular fragrances, that reduce or completely prevent the activity of the enzyme urease.

In a preferred embodiment according to the invention, the amount of the at least one fragrance is not sufficient to impart an inherent odor when added to a composition or product. The fragrances according to the invention have the advantage that, when used, in particular according to the use according to the invention, they are already active for the purposes of the present invention in such low concentrations at which no or at least no perceptible intrinsic odor of the fragrances is imparted. This allows these fragrances to be combined with different fragrances (not according to the invention) as well as optionally further components of a perfume oil, to produce particularly advantageous fragrance mixtures or to additionally perfume products with any desired scent.

In a preferred embodiment according to the invention, the composition or product is a consumer product. “Consumer products” according to the present invention are products that are bought by individuals or households for personal use. In other words, consumer products are goods that are bought for consumption by the average consumer. Preferred consumer products according to the present invention are personal care, in particular toilet care, in particular perfumes and/or fragrance compositions, home care, pet care, in particular cat litters and cosmetic products.

In a preferred embodiment according to the invention, the at least one fragrance is selected from fragrances having a molecular weight between 95 to 225 g/mol, in particular alcohols, carbonyls, aldehydes, ketones, ethers and esters. More preferably, the at least one fragrance is selected from fragrances having a molecular weight between 95 to 225 g/mol, in particular α,β-unsaturated carbonyls, β-unsaturated alcohols and γ-unsaturated esters.

In a further preferred embodiment according to the invention, the at least one fragrance is selected from the group consisting of cis-1-(1-Ethoxyethoxy)-3-hexene (leaf acetal), 2,6-Octadien-1-ol 3,7-dimethyl (mixed or isolated isomers; Geraniol), trans-2-hexenol, cis-3-hexenol, trans-2-hexenal, 3,7-dimethylocta-2,6-dienal (Citral FF), cis-3-hexenyl butyrate, (E)-1-(2,6,6-trimethyl-1-cyclohexa-1,3-dienyl)but-2-en-1-one (Damascenone total), (E)-1-(2,6,6-trimethyl-1-cyclohex-2-enyl)but-2-en-1-one (Damascone alpha), cis-3-hexenyl propionate, cis-3-hexenyl acetate, cis-3-hexenyl hexanoate, cis-3-hexenyl isovalerate, cis-3-hexenyl isobutyrate, (2E)-3-phenylprop-2-en-1-ol (cinnamic alcohol), alpha-methyl cinnamaldehyde, cis-3-hexenyl salicylate, 4-Hydroxy-2,5-dimethyl-3-furanone (Furaneol), methyl cyclopentenolone-3,2,2, methyl ethyl hydroxyfuranone, 2-ethoxy-4-methylphenol (Ultravanil), 2-Methoxyphenol (Guaiacol), 2-Methoxy-4-(prop-2-en-1-yl)phenol (Eugenol), 3-(1,3-Benzodioxol-5-yl)-2-methylpropanal (Helional), dimethyl-3,6-benzo-2(3H)-furanone (Furaminton/Tonkalactone™), (2Z)-3,7-dimethylocta-2,6-dien-1-ol (Nerol 900), 2,6-nonadien-1-ol, methyl ionone alpha, methyl ionone gamma, or mixtures thereof.

In a more preferred embodiment according to the invention, the at least one fragrance is selected from the group consisting of cis-3-hexenyl salicylate, cis-1-(1-Ethoxyethoxy)-3-hexene (leaf acetal), trans-2-hexenal, methyl ethyl hydroxyfuranone, cis-3-hexenyl butyrate, or mixtures thereof More preferred, the at least one fragrance is selected from trans-2-hexenal and/or methyl ethyl hydroxyfuranone. Most preferred, the at least one fragrance is selected from trans-2-hexenal.

In a further preferred embodiment, the at least one fragrance is selected from the group consisting of 2,6-Octadien-1-ol 3,7-dimethyl (mixed or isolated isomers; Geraniol) and/or 3,7-dimethylocta-2,6-dienal (Citral FF).

In other words, in a further preferred embodiment according to the invention, the at least one fragrance is selected from the group consisting of

| Name | Structure |
|---|---|
| cis-1-(1- Ethoxyethoxy)-3- hexene (Leaf acetal) | 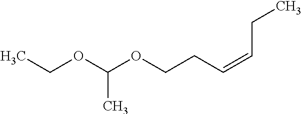 |
| 2,6-Octadien- 1-ol 3,7- dimethyl (mixed or isolated isomers; Geraniol) | 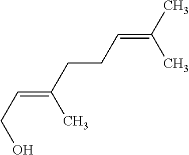 |
| trans-2-hexenol | 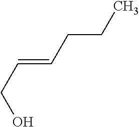 |
| cis-3-hexenol | 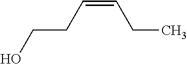 |
| trans-2-hexenal | 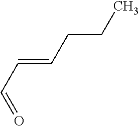 |
| 3,7- dimethylocta-2,6- dienal (Citral FF) | 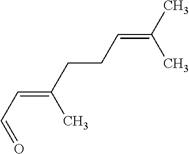 |
| cis-3-hexenyl butyrate | 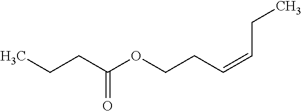 |
| (E)-1-(2,6,6- trimethyl-1- cyclohexa-1,3- dienyl)but-2- en-1-one (Damascenone total) | 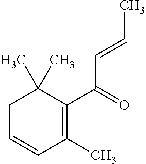 |
| (E)-1-(2,6,6- trimethyl-1- cyclohex-2- enyl)but-2-en- 1-one (Damascone alpha) | 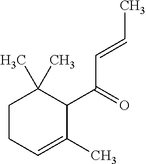 |
| cis-3-hexenyl propionate | 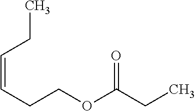 |
| cis-3-hexenyl acetate | 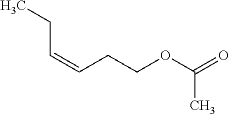 |
| cis-3-hexenyl hexanoate | 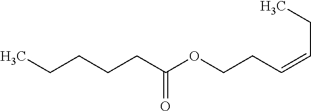 |
| cis-3-hexenyl isovalerate | 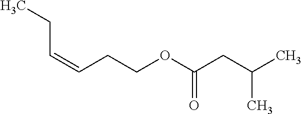 |
| cis-3-hexenyl isobutyrate | 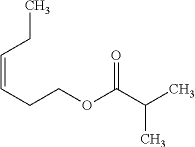 |
| (2E)-3-phenylprop- 2-en-1- ol (Cinnamic alcohol) | 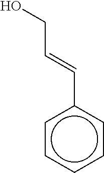 |
| alpha-methyl cinnamaldehyde | 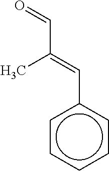 |
| cis-3-hexenyl salicylate | 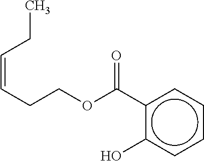 |
| 4-Hydroxy- 2,5-dimethyl-3- furanone (Furaneol) | 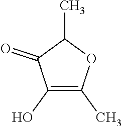 |
| methyl cyclopentenolone- 3,2,2 | 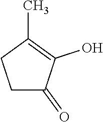 |
| methyl ethyl hydroxyfuranone | 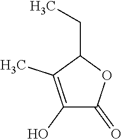 |
| 2-ethoxy-4- methylphenol (Ultravanil) | 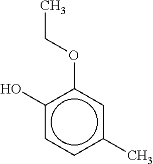 |
| 2-Methoxyphenol (Guaiacol) | 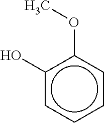 |
| 2-Methoxy- 4-(prop-2-en-1- yl)phenol (Eugenol) | 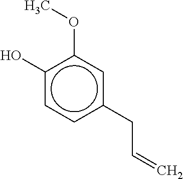 |
| 3-(1,3-Benzodioxol- 5-yl)-2- methylpropanal (Helional) | 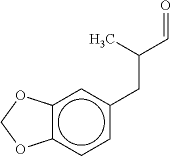 |
| dimethyl-3,6- benzo-2(3H)- furanone (Furaminton/ Tonkalactone ™) | 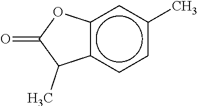 |
| (2Z)-3,7- dimethylocta-2,6- dien-1-ol (Nerol 900) | 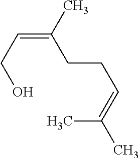 |
| 2,6-nonadien-1-ol | 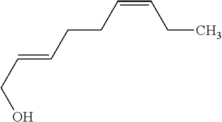 |
| Methyl ionone alpha | 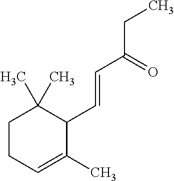 |
| Methyl ionone gamma | 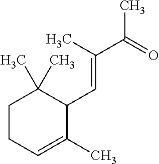 |

In another embodiment according to the invention, the at least one fragrance is used in an amount of from 0.01 to 10 weight percent when added to a composition or product, calculated on the total weight of the composition or product. More preferably, the at least one fragrance is used in an amount of from 0.05 to 5 weight percent; more preferably in an amount of from 0.05 to 1 weight percent and most preferably in an amount of from 0.05 to 0.5 weight percent; all weight percentages calculated on the total weight of the composition or product.

Particularly advantageous is a use according to the invention in which the ratio of the total mass of the at least one fragrance to the total mass of at least one precursor of ammonia is from 1:1 to 1:5, more preferably from 1:2 to 1:4.

A “precursor” according to the present invention is a compound that participates in a chemical reaction that produces another compound. In the present case, a “precursor of ammonia” is a compound that participates in a chemical reaction that produces ammonia. In one embodiment according to the invention, the precursor of ammonia is urea.

Thus, in a preferred embodiment according to the invention, the at least one fragrance is used as urease inhibitor in a composition or product, in particular in consumer products, together with at least one precursor of ammonia. Therefore, it is possible, when getting in contact with, for example, bacterial ureases, to completely, but at least partially, prevent the formation of ammonia by means of the fragrances of the present invention.

It goes without saying that one or more fragrances having a molecular weight between 95 to 225 g/mol can be used to inhibit urease activity. Therefore, for example one, two, three, four, five, six, seven, eight, nine, ten fragrances having a molecular weight between 95 to 225 g/mol can be used to inhibit urease activity in a consumer product.

Examples of other fragrances which can in principle be advantageously used as a further component together with the at least one fragrance having a molecular weight between 95 to 225 g/mol according to the invention, can be found, for example, in S. Arctander, Perfume and Flavor Chemicals, Vol. I and II, Montclair, N.J., 1969, Selbstverlag oder H. Surburg, J. Panten, Common Fragrance and Flavor Materials, 5th Ed., Wiley-VCH, Weinheim 2006.

Another embodiment of the present invention relates to a consumer product, comprising
(a) a fragrance composition comprising at least one fragrance having a molecular weight between 95 to 225 g/mol as a urease inhibitor; and
(b) at least one precursor of ammonia.

As already explained above, the fragrances which have a molecular weight between 95 to 225 g/mol inhibit the activity of urease, in particular bacterial urease, thereby reducing the amount of ammonia generated from ammonia precursors, as for example, urea.

In a preferred embodiment according to the invention, the consumer product further comprises
(c) at least one emulsifier selected from the group consisting of Cetearyl Ethylhexanoate, Cetearyl Alcohol, Potassium cetyl phosphate, Sodium cetyl sulfate, PEG-40 Stearate.

In a preferred embodiment according to the invention, the consumer product does not contain any further urease inhibitor. As stated above, preferred consumer products according to the present invention are personal care, in particular toilet care, in particular perfumes and/or fragrance compositions, home care, pet care, in particular cat litters and cosmetic products.

Preferably, the at least one fragrance is present in an amount of from 0.01 to 10 weight percent, calculated on the total weight of the consumer product. More preferably, the at least one fragrance is present in an amount of from 0.05 to 5 weight percent, calculated on the total weight of the consumer product. Even more preferably, the at least one fragrance is present in an amount of from 0.05 to 1 weight percent, calculated on the total weight of the consumer product.

In a preferred embodiment, the ratio between the at least one fragrance having a molecular weight between 95 to 225 g/mol and the at least one precursor of ammonia is from 1:1 to 1:5, more preferably from 1:2 to 1:4.

In a further preferred embodiment according to the invention, the at least one fragrance is selected from the group consisting of cis-1-(1-Ethoxyethoxy)-3-hexene (leaf acetal), 2,6-Octadien-1-ol 3,7-dimethyl (mixed or isolated isomers; Geraniol), trans-2-hexenol, cis-3-hexenol, trans-2-hexenal, 3,7-dimethylocta-2,6-dienal (Citral FF), cis-3-hexenyl butyrate, (E)-1-(2,6,6-trimethyl-1-cyclohexa-1,3-dienyl)but-2-en-1-one (Damascenone total), (E)-1-(2,6,6-trimethyl-1-cyclohex-2-enyl)but-2-en-1-one (Damascone alpha), cis-3-hexenyl propionate, cis-3-hexenyl acetate, cis-3-hexenyl hexanoate, cis-3-hexenyl isovalerate, cis-3-hexenyl isobutyrate, (2E)-3-phenylprop-2-en-1-ol (cinnamic alcohol), alpha-methyl cinnamaldehyde, cis-3-hexenyl salicylate, 4-Hydroxy-2,5-dimethyl-3-furanone (Furaneol), methyl cyclopentenolone-3,2,2, methyl ethyl hydroxyfuranone, 2-ethoxy-4-methylphenol (Ultravanil), 2-Methoxyphenol (Guaiacol), 2-Methoxy-4-(prop-2-en-1-yl)phenol (Eugenol), 3-(1,3-Benzodioxol-5-yl)-2-methylpropanal (Helional), dimethyl-3,6-benzo-2(3H)-furanone (Furaminton/Tonkalactone™), (2Z)-3,7-dimethylocta-2,6-dien-1-ol (Nerol 900), 2,6-nonadien-1-ol, methyl ionone alpha, methyl ionone gamma, or mixtures thereof.

In a more preferred embodiment according to the invention, the at least one fragrance is selected from the group consisting of cis-3-hexenyl salicylate, cis-1-(1-Ethoxyethoxy)-3-hexene (leaf acetal), trans-2-hexenal, methyl ethyl hydroxyfuranone, cis-3-hexenyl butyrate or mixtures thereof. More preferred, the at least one fragrance is selected from trans-2-hexenal and/or methyl ethyl hydroxyfuranone. Most preferred, the at least one fragrance is selected from trans-2-hexenal.

In a further preferred embodiment, the at least one fragrance is selected from the group consisting of 2,6-Octadien-1-ol 3,7-dimethyl (mixed or isolated isomers; Geraniol) and/or 3,7-dimethylocta-2,6-dienal (Citral FF).

Fragrance compositions according to the invention, which contain the at least one fragrance having a molecular weight between 95 to 225 g/mol can be in liquid form, undiluted or diluted with a solvent and is advantageously used for perfuming. Preferred solvents for this purpose are ethanol, isopropanol, diethylene glycol monoethyl ether, glycerol, propylene glycol, 1,2-butylene glycol, dipropylene glycol, diethyl phthalate, triethyl citrate, isopropyl myristate, triacetin and diacetin.

Furthermore, fragrance compositions according to the invention may be adsorbed on a carrier that provides both fine distribution of the fragrances in the product and controlled release upon application. Such carriers may be porous inorganic materials such as light sulfate, silica gels, zeolites, gypsums, clays, clay granules, aerated concrete, etc., or organic materials such as woods, cellulose-based materials, sugars, dextrins (e.g., maltodextrin), or plastics such as PVC, polyvinyl acetates, or polyurethanes. The resulting combination of composition according to the invention and carrier is also to be understood as a fragrance composition according to the invention, or may be present as an article according to the invention (as further described herein).

Fragrance compositions according to the invention may also be microencapsulated, spray-dried, as inclusion complexes, or as extrusion products, and may be added in this form, for example, to a product to be perfumed.

If necessary, the properties of the compositions can be further optimized by so-called “coating” with suitable materials with a view to more targeted fragrance release, for which purpose waxy plastics such as polyvinyl alcohol are preferably used.

Other preferred embodiments and definitions which have been described above also apply, of course, to the consumer product according to the invention.

A further embodiment of the invention relates to a method for inhibiting urease activity in a composition or product, wherein at least one fragrance having a molecular weight between 95 to 225 g/mol is added to the composition or product.

Preferably, the at least one fragrance is added in an amount of from 0.01 to 10 weight percent, calculated on the total weight of the consumer product. More preferably, the at least one fragrance is added in an amount of from 0.05 to 5 weight percent, calculated on the total weight of the consumer product. Even more preferably, the at least one fragrance is added in an amount of from 0.05 to 1 weight percent, calculated on the total weight of the consumer product.

Further preferably, the composition or product is a consumer product.

The above-mentioned preferred embodiments and definitions of course also apply to the method according to the present invention.

A further embodiment of the invention relates to a cat litter comprising at least one fragrance having a molecular weight between 95 to 225 g/mol as a urease inhibitor.

All above-mentioned preferred embodiments and definitions of course also apply to the cat litter according to the present invention.

In the following, embodiments of the invention are further characterized.

### EXAMPLES

### Screening of Urease Inhibition

Urease catalyzed ammonia formation leads to a change in pH (alkalization). Hence the reaction can be monitored using a pH indicator. The enzymatic screening assay is described below. The colorimetric assay is conducted in 96 well plates in combination with a microplate spectrophotometer with Phenol Red as pH sensitive indicator.

In brief: the substrate urea, the fragrance raw material and the urease are mixed in the wells and the change in absorption at 560 nm is monitored for 1 h. The resulting absorbance curves are compared to untreated samples (negative controls, without inhibitors) and samples with commercially available NBPT inhibitor (positive control).

TABLE 1
Composition of wells

| Compound | Concentration | |
|---|---|---|
| PBS Buffer | 1x * | |
| Additive (fragrance, | 0.1 | w/w |
| inhibitor) | | |
| Urea | 0.2% | w/w |
| Urease | 0.5 | U/ml |

* Concentration of PBS buffer components: NaCl 0.14M; KCl, 0.003M; Na2HPO4, 0.01M; KH2PO4 0.002M. The buffer concentrate is pH adjusted with aqueous HCl for pH 7.4.

The rate of reaction is derived from the color change (Δ Absorbance at 560 nm) over time:

$$Δ Absorbance = \frac{Absorbance {t}_{1} - Absorbance {t}_{0}}{{t}_{1} - {t}_{0}}$$

The inhibition efficacy is then calculated as follows:

$$I = 1 - ( \frac{Δ Absorbance of sample}{Δ Absorbance of negative control} )$$

### Identification of Positive Hits

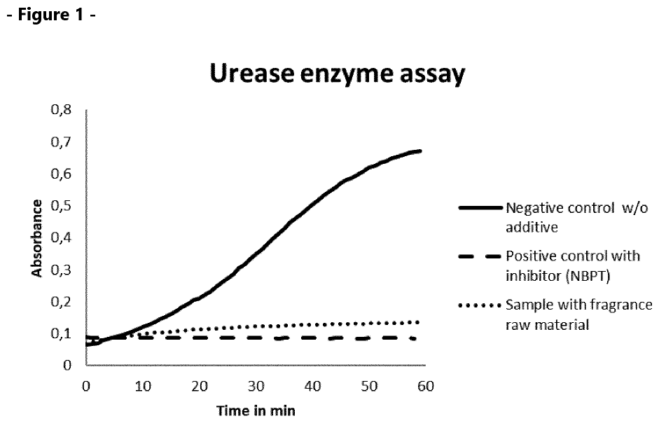

FIG. 1 represents a typical result from the screening assay. The corresponding calculations are shown in table 2. In this case, the fragrance raw material (green trace) shows a significant inhibition of the enzyme urease.

TABLE 2
Results screening assay

| | Δ Absorbance at | |
|---|---|---|
| Samples | 1 h (560 nm) | Inhibition |
| Negative control, | 0.61 | 0 |
| w/o additive | | |
| Positive control, | 0.00 | 1.00 |
| with inhibitor | | |
| (NBPT) | | |
| Sample with | 0.07 | 0.89 |
| fragrance raw | | |
| material | | |

### Positive Hits

A total of 29 highly active compounds could be identified through in vitro screening. Interestingly, they share common structural features like a molecular weight between 95 to 225 g/mol, α,β-unsaturated carbonyls (e.g. Damascones and Ionones), a range of β-unsaturated alcohols and γ-unsaturated esters.

| Material | Name: global/english | Inhibition |
|---|---|---|
| 131415 | LEAF ACETAL | 0.964 |
| 903290 | GERANIOL 60 B | 0.910 |
| 130870 | HEXENOL TRANS-2 | 0.912 |
| 131104 | HEXENOL CIS-3 | 0.888 |
| 131080 | HEXENAL TRANS-2 | 0.961 |
| 197010 | CITRAL FF | 2.145 |
| 131389 | HEXENYL BUTYRATE CIS-3 | 0.999 |
| 107047 | DAMASCENONE TOTAL | 0.985 |
| 131354 | DAMASCONE ALPHA | 0.991 |
| 173272 | HEXENYL PROPIONATE CIS-3 | 0.956 |
| 131185 | HEXENYL ACETATE CIS-3 | 0.723 |
| 131189 | HEXENYL CAPROATE CIS-3 | 0.824 |
| 131255 | HEXENYL ISOVALERATE CIS-3 | 0.724 |
| 657925 | HEXENYL ISOBUTYRATE CIS-3 | 0.865 |
| 654060 | CINNAMIC ALCOHOL | 0.931 |
| 613155 | METHYL CINNAMIC ALDEHYDE ALPHA | 0.993 |
| 600301 | HEXENYL SALICYLATE CIS-3 | 0.989 |
| 131225 | FURANEOL | 0.985 |
| 130573 | METHYL CYCLOPENTENOLONE-3,2,2 | 0.992 |
| | NAT. | |
| 131396 | METHYL ETHYL HYDROXYFURANONE | 1.000 |
| 125801 | ULTRAVANIL | 0.786 |
| 164364 | GUAIACOL | 0.718 |
| 608025 | EUGENOL NAT. | 0.908 |
| 103828 | HELIONAL | 1.012 |
| 600291 | FURAMINTON/TONKALACTONE ™ | 0.669 |
| 103885 | NEROL 900 | 0.758 |
| 131091 | NONADIENOL-2,6 | 0.885 |
| 103236 | METHYL IONONE ALPHA/IFF | 0.936 |
| 103927 | METHYL IONONE GAMMA PURE/IFF | 0.942 |

### Application in Cat Litter

The efficiency of reducing ammonia formation in cat litter was tested with different fragrance raw materials that are shown in the table below (table 3).

In brief, the litter was coated with a final concentration of 0.1% (w/w) of active material respective to the dry litter material. In a glass bottle with a total volume of 5 l, the litter is treated with artificial cat urine that consists of an aqueous salt solution containing urea and urease. The development of ammonia is measured through headspace sampling with an ammonia sensitive detector over a period of 5 days.

TABLE 3
Different fragrances tested in cat litter

| | Relative ammonia |
|---|---|
| Sample | formation in % |
| Hexenyl salicylate cis-3 | 65 |
| Leaf acetal | 81 |
| Hexenal trans-2 | 32 |
| Methylethyl hydroxyfuranone | 41 |
| Hesenyl butyrate cis-3 | 65 |
| Negative control (w/o additive) | 100 |

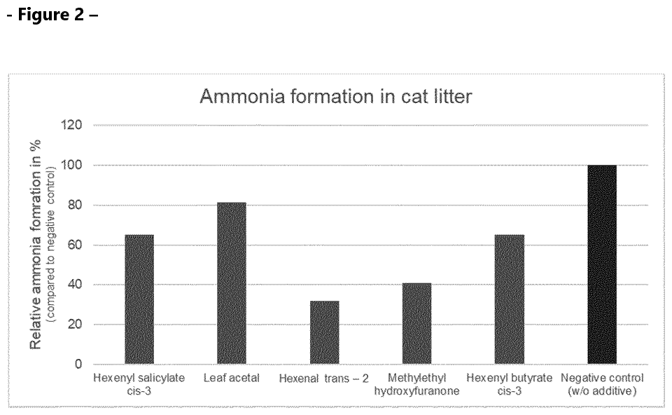

The results confirm the urease inhibitory effect of fragrance raw materials with all tested fragrances showing a less relative ammonia formation as compared to the negative (untreated) control. FIG. 2 also shows the results.

1. Use of at least one fragrance having a molecular weight between 95 to 225 g/mol as a urease inhibitor.

2. The use according to claim 1, wherein the amount of the at least one fragrance is not sufficient to impart an inherent odor when added to a composition or product.

3. The use according to claim 1 and/or 2, wherein the at least one fragrance is selected from the group consisting of cis-1-(1-Ethoxyethoxy)-3-hexene (leaf acetal), 2,6-Octadien-1-ol 3,7-dimethyl (mixed or isolated isomers; Geraniol), trans-2-hexenol, cis-3-hexenol, trans-2-hexenal, 3,7-dimethylocta-2,6-dienal (Citral FF), cis-3-hexenyl butyrate, (E)-1-(2,6,6-trimethyl-1-cyclohexa-1,3-dienyl)but-2-en-1-one (Damascenone total), (E)-1-(2,6,6-trimethyl-1-cyclohex-2-enyl)but-2-en-1-one (Damascone alpha), cis-3-hexenyl propionate, cis-3-hexenyl acetate, cis-3-hexenyl hexanoate, cis-3-hexenyl isovalerate, cis-3-hexenyl isobutyrate, (2E)-3-phenylprop-2-en-1-ol (cinnamic alcohol), alpha-methyl cinnamaldehyde, cis-3-hexenyl salicylate, 4-Hydroxy-2,5-dimethyl-3-furanone (Furaneol), methyl cyclopentenolone-3,2,2, methyl ethyl hydroxyfuranone, 2-ethoxy-4-methylphenol (Ultravanil), 2-Methoxyphenol (Guaiacol), 2-Methoxy-4-(prop-2-en-1-yl)phenol (Eugenol), 3-(1,3-Benzodioxol-5-yl)-2-methylpropanal (Helional), dimethyl-3,6-benzo-2 (3H)-furanone (Furaminton/Tonkalactone™), (2Z)-3,7-dimethylocta-2,6-dien-1-ol (Nerol 900), 2,6-nonadien-1-ol, methyl ionone alpha, methyl ionone gamma, or mixtures thereof.

4. The use according to at least one of the preceding claims, wherein the at least one fragrance is selected from the group consisting of cis-3-hexenyl salicylate, cis-1-(1-Ethoxyethoxy)-3-hexene (leaf acetal), trans-2-hexenal, methyl ethyl hydroxyfuranone, cis-3-hexenyl butyrate or mixtures thereof.

5. The use according to at least one of the preceding claims, wherein the at least one fragrance is selected from trans-2-hexenal and/or methyl ethyl hydroxyfuranone

6. The use according to at least one of the preceding claims, wherein the at least one fragrance is selected from trans-2-hexenal.

7. The use according to at least one of the preceding claims, wherein the amount of the at least one fragrance is of from 0.01 to 10 weight percent when added to a composition or product, calculated on the total weight of the composition or product.

8. A consumer product, comprising
(a) a fragrance composition comprising at least one fragrance having a molecular weight between 95 to 225 g/mol as an urease inhibitor; and
(b) at least one precursor of ammonia.

9. The consumer product according to claim 8, wherein the consumer product does not contain any further urease inhibitor.

10. The consumer product according to claim 8 and/or 9, wherein the at least one fragrance is present in an amount of from 0.01 to 10 weight percent, calculated on the total weight of the consumer product.

11. The consumer product according to claim 8 and/or 9, wherein the at least one fragrance is present in an amount of from 0.05 to 1 weight percent, calculated on the total weight of the consumer product.

12. The consumer product according to claims 8 to 11, wherein the ratio between the at least one fragrance having a molecular weight between 95 to 225 g/mol and the at least one precursor of ammonia is from 1:1 to 1:5.

13. A method for inhibiting urease activity in a composition or product, wherein at least one fragrance having a molecular weight between 95 to 225 g/mol is added to the composition or product.

14. The method according to claim 13, wherein the at least one fragrance is added in an amount of from 0.01 to 10 weight percent, calculated on the total weight of the composition or product.

15. A cat litter comprising at least one fragrance having a molecular weight between 95 to 225 g/mol as an urease inhibitor.
